(19) United States
(12) Patent Application Publication
(10) Pub. No.: US 2019/0175796 A1
(43) Pub. Date: Jun. 13, 2019

(54) PHOTOINITIATING POLYMERISABLE COMPOSITION

(71) Applicant: LANCASTER UNIVERSITY BUSINESS ENTERPRISES LIMITED, Lancaster (GB)

(72) Inventor: John George HARDY, Lancaster (GB)

(21) Appl. No.: 16/323,015

(22) PCT Filed: Aug. 1, 2017
(86) PCT No.: PCT/GB2017/052235

§ 371 Date: Feb. 4, 2019

(30) Foreign Application Priority Data
Aug. 5, 2016 (GB) 1613540.2

(51) Int. Cl. A61L 31/06; A61L 31/14; A61N 1/05

(52) U.S. Cl. CPC A61L 31/06; A61L 31/14; A61L 2400/06; A61L 2300/442; A61N 1/0504

(57) ABSTRACT

The present invention provides a method of producing a visual marking on the exterior of a human or animal body. There is also provided a method of forming a polymer within a human or animal body. The resultant polymer generally has an electrical conductivity of 10⁻¹⁰ S/cm or more.

The present invention relates to an injectable composition. An in vivo method of forming a polymeric structure, such as a medical device, is also described. There is also provided a method of medical treatment including the implantation of the composition to a human or animal body, and the subsequent polymerization of the composition within the human or animal body.

### BACKGROUND TO THE INVENTION

It is known to use multi-photon photo-polymerization ex vivo to build a structure modelled on a biological organ (for example see US2013197668A1). A solid block of gel containing monomers and a 2-photon active photo-initiator is prepared as a raw material. The shape of a desired object is traced out by the control of laser beams, optionally there may be additional steps such as a fixing step, and then excess gel is washed away to leave the desired structure.

In one type of photo-polymerization, light is absorbed directly by a reactant monomer. In a second type of photo-polymerization, light is absorbed by a photo-initiator material which absorbs the light and then transfers energy to the monomer to effect polymerization.

It is known to use multi-photon techniques destructively to induce controlled release of drugs from a structure in vivo (see for example US2003191458A1).

Currently the provision of structures in vivo requires incision and implantation. The present invention is advantageous because it permits in situ in vivo fabrication of structures and may require as few as one implantation (suitably one injection) of the precursor materials.

### STATEMENT OF INVENTION

A method of forming a polymer within a human or animal body comprising:
implanting a composition at a site in the human or animal body wherein the composition comprises a polymerisable precursor and a photo-initiator wherein the photo-initiator causes the polymerisable precursor to polymerise where the photo-initiator is in its excited state;
illuminating the site with electromagnetic radiation having a first wavelength of 400 to 1600 nm, wherein absorption of two or more photons of the electromagnetic radiation excites the photo-initiator causing initiation of polymerisation of the polymerisable precursor to form a polymer within the human or animal body, wherein polymerisation causes a change in physical state of the polymerisable precursor;
wherein the polymer has an electrical conductivity of 10⁻¹⁰ S/cm to 10⁴ S/cm or more.

Generally, the change in physical state involves solidification of the polymerisable precursor. Alternatively, the viscosity of the polymerisable precursor may be greatly increased, for example to yield a gel (including a hydrogel), paste, foam or plastic.

According to a further aspect of the present invention, there is provided a method of producing a visual marking on the exterior of a human or animal body comprising:

implanting a composition at a site in the human or animal body 1 cm or less from the exterior of the human or animal body wherein the composition comprises a polymerisable precursor and a photo-initiator wherein the photo-initiator causes the polymerisable precursor to polymerise where the photo-initiator is in its excited state;

illuminating the site with electromagnetic radiation having a wavelength of 400 to 1600 nm, wherein absorption of two or more photons of the electromagnetic radiation excites the photo-initiator causing initiation of polymerisation of the polymerisable precursor to form a polymer within the human or animal body, wherein the polymer is visible on the exterior of the human or animal body.

Generally, the visual marking is in the form of a tattoo.

According to one embodiment, the composition includes second photo-initiatable functional moieties which become excited at a different wavelength to the photo-initiator and wherein the polymer may be dis-assembled through illumination of the site with electromagnetic radiation having a second wavelength. The second wavelength may be at least 200 nm higher or lower than the first wavelength wherein absorption of two or more photons of the electromagnetic radiation causes the second photo-initiatable functional moieties on the polymer to become excited and destroy the polymer.

According to an aspect of the present invention, there is provided an injectable composition comprising a polymerisable precursor and a photo-initiator wherein the photo-initiator causes the polymerisable precursor to polymerise where the photo-initiator is in its excited state,
wherein absorption of two or more photons of electromagnetic radiation having a wavelength of 400 to 1600 nm excites the photo-initiator causing initiation of polymerisation of the polymerisable precursor,
wherein the resultant polymer has an associated electrical conductivity of at least 10⁻¹⁰ S/cm to 10⁴ S/cm or more.

According to a further aspect of the present invention, there is provided a buffered aqueous based composition comprising monomers that are derivatives of pyrrole, aniline, thiophene, 3,4-ethylenedioxythiophene, fluorene, phenylene, pyrene, azulene, napthalene, indole, azepine, p-phenylene sulfide, p-phenylene vinylene, and/or furans; and moreover, a photo-initiator, and dye(s).

According to a further aspect of the present invention there is provided a buffered aqueous based composition comprising pyrrole, a photo-initiator (generally irgacure) and a dye.

According to a further aspect of the present invention there is provided a method of forming a polymeric structure within a human or animal body utilising the methods as described herein.

According to a further aspect of the present invention, there is provided a composition as described herein for use in therapy.

According to a further aspect of the present invention, there is provided a composition as described herein for use in the treatment of conditions that respond to electricity (i.e. using the polymer deposited as an electrode to stimulate the tissue surrounding it for deep brain stimulation, for electrical stimulation of neurons, stem cells or muscle cells), or prevention of cell migration (cancer cells, bacteria, etc), or the delivery of active ingredients (drugs) entrapped within the polymer matrix.

There is also provided a method of medical treatment comprising forming a polymer within a human or animal body in accordance with the method described herein.

In yet another aspect, the present teachings include a kit, where the kit includes one or more of the compositions of the present teachings, or the components necessary to form the compositions of the present invention, and instructions for use thereof. Generally, the kit would comprise an energy source.

According to a further embodiment, there is provided a system for performing the methods disclosed herein. The system can include the composition as disclosed herein and one or more energy sources, typically lasers. The system may include an analytical instrument used to measure the wavelength and intensity of the energy source(s), and/or to measure the amount of composition implanted in the human or animal body. The system also can include a suitably programmed computer for carrying out one or more steps of the methods. For example, the suitably programmed computer can carry out or assist in one or more of control of the intensity of the energy source(s), measuring any fluctuations in the intensity of the energy source(s), and equivalents thereof.

Throughout the Application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the Application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds and therapeutic combinations of the present teachings can be useful for treating a pathological condition or disorder in a patient, for example, a human. As used herein, “treating” refers to partially or completely alleviating and/or ameliorating the condition and/or symptoms thereof. The present teachings accordingly include a method of providing to a patient a pharmaceutical composition that includes a compound or therapeutic combination of the present teachings in combination or association with a pharmaceutically acceptable carrier. Compounds and therapeutic combinations of the present teachings can be administered alone or in combination with other therapeutically effective compounds or therapies for the treatment of a pathological condition or disorder.

As used herein, “patient” refers to a human, animal, fish or bird, in particular a mammal, such as a human although mention may also be made of primates, dogs, cats, rats, horses, camels, farm livestock such as cattle sheep, pigs, goats, deer; birds including chicken, geese, birds of prey and endangered animals including endangered birds.

Because the present teachings provide pharmaceutical formulations and their intended use is with patients such as humans, each of the ingredients or compounds of a pharmaceutical formulation described herein can be a pharmaceutically acceptable ingredient or compound.

As used herein, a “compound” refers to the compound itself and its pharmaceutically acceptable salts and hydrates unless otherwise understood from the context of the description or expressly limited to one particular form of the compound, i.e., the compound itself, or a pharmaceutically acceptable salt or hydrate thereof.

Features, integers, characteristics, described in conjunction with a particular aspect, embodiment or example of the invention are to be understood to be applicable to any other aspect, embodiment or example described herein unless incompatible therewith.

### DETAILED DESCRIPTION

Method

A method of forming a polymer within a human or animal body comprising:
implanting a composition at a site in the human or animal body wherein the composition comprises a polymerisable precursor and a photo-initiator wherein the photo-initiator causes the polymerisable precursor to polymerise where the photo-initiator is in its excited state;
illuminating the site with electromagnetic radiation having a first wavelength of 400 to 1600 nm, wherein absorption of two or more photons of the electromagnetic radiation excites the photo-initiator causing initiation of polymerisation of the polymerisable precursor to form a polymer within the human or animal body, wherein polymerisation causes a change in physical state of the polymerisable precursor;
wherein the polymer has an electrical conductivity of 10⁻¹⁰ S/cm to 10⁴ S/cm or more.

Generally, the change in physical state involves solidification of the polymerisable precursor. Alternatively, the viscosity of the polymerisable precursor may be greatly increased, for example to yield a gel (including a hydrogel), paste, foam or plastic. Advantageously, the polymerisation causes solidification of the polymerisable precursor.

Generally, the dimensions of the polymerised area (in general the solidified area) are at least 1 nm, typically at least 100 nm. Typically, the polymerised area has an associated thickness of less than 1 cm.

Currently the provision of structures in vivo requires incision and implantation. The present invention is advantageous because it permits in situ in vivo fabrication of structures and may require as few as one injection of the precursor materials.

The use of multi-photon technology is advantageous because it may be controlled so that a desired photochemical reaction occurs only at the point of intersection of two or more photon beams. For example two-photon polymerisation may provide point by point scanning to build a desired structure.

According to one aspect of the present invention, at least one of the beams may be scanned so as to produce a pattern.

The composition is generally implanted through injection. The composition may be injected into the lower layers of the skin, or subcutaneously. The composition may be implanted via bolus injection. Alternatively, the composition may be implanted through the use of a catheter.

Generally the composition is implanted 10 cm or less from the surface of the human or animal body, typically 5 cm or less, suitably 1 cm or less, more suitably within 5 mm of the surface of the human or animal body.

According to one embodiment, the composition is implanted to the lower layers of skin, or directly under the skin of the human or animal body. Alternatively, the composition may be implanted directly under the surface of the eye of the human or animal. Suitably the composition may be injected into other tissue, for example muscle or fat, or around an organ of the human or animal body.

Alternatively, the composition may be implanted further from the surface of the human or animal body. The electromagnetic radiation source may be inserted into the body to illuminate the site of the implanted composition (suitably through keyhole surgery).

The closer the site of the implanted composition to the energy source upon illumination of the site by the energy source, the more targeted and controllable the polymerisation of the composition.

Generally, the site of the implanted composition should be no more than 30 cm from the energy source upon illumination of the site, suitably 10 cm or less from the energy source. Typically the site is 5 cm or less from the energy source upon illumination, suitably 1 cm or less from the energy source upon illumination.

To excite the photo-initiator, a threshold amount of radiation must be absorbed. The wavelength of the incident radiation is generally 400 to 1600 nm.

The threshold incident radiation is generally in the form of at least two photons from intersecting photon beams.

Generally, the composition is subjected to electromagnetic radiation from outside the human or animal body to initiate polymerisation of the monomer.

Absorption by the photo-initiator of a threshold amount of electromagnetic radiation excites the photo-initiator, and the excited photo-initiator initiates polymerisation of the polymerisable precursor. Generally, absorption of electromagnetic radiation from the intersection of two or more photon beams is required to excite the photo-initiator, meaning that the photochemical reaction is very controllable and predictable.

The use of multi-photon technology is advantageous because it may be controlled so that a desired photochemical reaction occurs only at the point of intersection of two or more photon beams. For example, two-photon polymerisation may provide point by point scanning to build a desired structure.

Upon polymerisation, the polymerisable precursor undergoes a change in physical state. Generally the polymerisable precursor solidifies. Alternatively, a hydrogel or plastics material may be formed. Generally, the area of polymerisation, of the precursor is within 1,000,000 nm of the incident threshold electromagnetic radiation, typically within 10,000 nm of the intersection of two or more photon beams.

The electromagnetic radiation is generally emitted by an energy source such as a laser.

The dimensions of the polymer formed according to the method of the present invention (width, length, thickness) are generally at least 10 nm, typically 10 nm to 10 cm, suitably 50 nm to 5 cm thick, more suitably 50 nm to 1 cm, advantageously 50 nm to 100 nm.

Generally, the polymer formed is a relatively thin polymer film or coating, and has a relatively large associated length and width. The polymer generally has a thickness of 10 nm to 200 nm, (typically a maximum thickness of 100 nm) and a width and/or length of 1 cm to 5 cm.

According to one embodiment, the polymer formed according to the method of the present invention (width, length, thickness) may have dimensions of from 1 nm to 2 mm.

It is envisaged that the polymer may have dimensions of from 10 cm to 2 m.

Where the polymer has a thickness of more than 1 cm, areas of the composition furthest from an external surface of the human or animal body are generally polymerised before areas of the composition closer to an external surface of the human or animal body by controlling the intersection of the photon beams within the human or animal body.

In order to allow adequate control, any area of the composition to be polymerised is generally no more than 10 cm from an external surface of the human or animal body. Alternatively, the energy source may be inserted into the human or animal body to allow polymerisation of compositions at greater depths.

The composition may suitably include a visual marker such as a dye to assist medical practitioners such as surgeons as they are forming the polymer. The dye could become apparent as the polymerisable precursor polymerised, or may be visible in the polymerisable precursor but not in the polymer.

The polymer formed according to the method of the present invention may be in the form of a polymeric structure or medical device. According to one embodiment, cells from the human or animal body may colonise the polymeric structure formed over time.

The polymer formed has an electrical conductivity of at least 10⁻¹⁰ S/cm to 10⁴ S/cm, generally 1 S/cm to 5,000 S/cm, suitably 10 S/cm to 1,000 S/cm, and this allows potential uses in the fabrication of devices within the human or animal body.

Particular mention may be made of the use of a polymer formed in vivo in accordance with the method of the present invention in the formation of an electrode (including an electrode with a connection to an implanted device/chip), an antenna, an induction loop with connections to power an implanted device wirelessly, an electro-magnetic shield, a support structure, a radio, a hearing aid, ID tag or tattoo.

According to one embodiment, the polymer formed may be a stimulus responsive material. The physical dimensions of the polymer may alter in response to a stimulus, in particular the shape/size/properties (for instance mechanics, optical, electrical properties) may alter in response to the stimulus. The polymer may deliver drugs/cells in response to the stimulus.

Method of Producing a Visual Marking

According to a further aspect of the present invention, there is provided a method of producing a visual marking on the exterior of a human or animal body comprising:
implanting a composition at a site in the human or animal body 1 cm or less from the exterior of the human or animal body wherein the composition comprises a polymerisable precursor and a photo-initiator wherein the photo-initiator causes the polymerisable precursor to polymerise where the photo-initiator is in its excited state;
illuminating the site with electromagnetic radiation having a wavelength of 400 to 1600 nm, wherein absorption of two or more photons of the electromagnetic radiation excites the photo-initiator causing initiation of polymerisation of the polymerisable precursor to form a polymer within the human or animal body, wherein the polymer is visible on the exterior of the human or animal body.

The resultant polymer generally has an electrical conductivity of 10⁻¹⁰ S/cm to 10⁴ S/cm or more, suitably 0.0001 S/cm to 1,000 S/cm; typically, 0.001 S/cm to 1 S/cm.

The resultant polymer is generally one colour, although functional groups may be added to alter the colour of the polymer at different areas.

According to one embodiment, the resultant polymer shows more than one colour on the exterior of the human or animal body.

Generally, the visual marking is in the form of a tattoo.

The composition is suitably injected into the lower surfaces of the skin of the human or animal body. The visual marking may grow out as the skin is shed. Alternatively, the composition may be injected just under the skin of the human or animal body, to provide a permanent visual marking.

The visual marking may be altered or removed using the polymer dis-assembling methods of the present teachings. This can provide significant advantages over current tattooing methods.

The visual marking of the present invention may provide an alternative to branding animals. The visual marking of the present teachings may be altered or removed using the polymer dis-assembling methods of the present teachings, and this can prove useful if the ownership of the animal changes.

Method of Dis-Assembling Polymer

By use of suitable chemistry in the present invention, a first multi-photon reaction (using first functional moieties) may be used to assemble a structure; and a second multi-photon reaction at a different energy (using second functional moieties) may be used later to disassemble the same structure.

According to one embodiment, the composition includes second photo-initiatable functional moieties which become excited at a different wavelength to the photo-initiator. Generally the difference in wavelength is at least 200 nm. The polymerisable precursor generally includes the second photo-initiatable functional moieties, and following polymerisation thereof, the polymer generally comprises the second photo-initiatable functional moieties.

The polymer formed according to the method of the present teachings may be dis-assembled through illumination of the site with electromagnetic radiation having a second wavelength. The second wavelength may be at least 200 nm higher or lower than the first wavelength wherein absorption of two or more photons of the electromagnetic radiation causes the second photo-initiatable functional moieties on the polymer to become excited and destroy the polymer.

The method described herein may include dis-assembling the polymer formed by illuminating the site with electromagnetic radiation having a second wavelength. The second wavelength may be different to the first wavelength by at least 200 nm, wherein absorption of two or more photons of the electromagnetic radiation excites second photo-initiatable functional moieties on the polymer, and the excited second photo-initiatable functional moieties destroy the polymer.

As for the method of forming the polymer, the photo-initiatable destruction is precisely targeted and controllable. Generally, the destruction of the polymer is limited to 5 mm or less, typically 1 mm or less from the location of the incident electromagnetic radiation; typically, from the location of the intersection of two or more photon beams.

Upon destruction of the polymer through the reaction initiated through the excitation of the second photo-initiatable functional moieties, waste products are formed and these are generally non-toxic to the human or animal body. Generally, the waste products are bio-absorbable and may be absorbed by the human or animal body.

Suitable photo-initiatable functional moieties to destroy the polymer include: azobenzene (excitable at around 366 to 420 nm), o-nitrobenzyl (excitable at 350, 750 nm), 3,4-dihydroxycinnamic acid-co-4-hydroxycinnamic acid (P(3,4DHCA-co-4HCA)) (at 254, 280 nm), dipalmitoylphosphatidylcholine (at 514 nm), diazonaphthoquinone-based molecules (using UV/NIR).

Generally, the second photo-initiatable functional moieties are initiated at lower wavelengths compared to the wavelength for exciting the photo-initiator.

Composition

According to an aspect of the present invention, there is provided an injectable composition comprising a polymerisable precursor and a photo-initiator wherein the photo-initiator causes the polymerisable precursor to polymerise where the photo-initiator is in its excited state,
wherein absorption of two or more photons of electromagnetic radiation having a wavelength of 400 nm to 1600 nm excites the photo-initiator causing initiation of polymerisation of the polymerisable precursor,
wherein the resultant polymer has an associated electrical conductivity of at least 10⁻¹⁰ S/cm to 10⁴ S/cm or more.

The composition generally includes a visual indicator which alters upon polymerisation of the polymerisable precursor. Suitable visual indicators include dyes. Generally, the visual indicator is visible in the polymerisable precursor but not in the polymer. Alternatively, the visual indicator may be visible in the polymer but not the polymerisable precursor.

The visual indicator may be visible upon illumination with UV light.

The visual indicator may change colour between monomer and polymer.

The incorporation of a visual indicator which alters upon polymerisation of the polymerisable precursor is useful following implantation of the composition into a human or animal body. Specifically, it can assist in showing the precise location of the composition.

Generally said composition has a viscosity in the range from 1 centipoise (similar to water), to 250,000 centipoise (similar to peanut butter). A viscosity of 1 centipoise or more helps to ensure the injected composition is retained in a predictable position (generally at the site of injection). A viscosity of 250,000 centipoise or less reduces the risk of blockages within the human or animal body, which can be of paramount importance where the composition is implanted near a blood vessel.

Typically, the composition is buffered to a pH of 4-10, but most commonly pH 7.4. Generally, the composition is sterilised prior to implantation.

The polymerisable precursor may comprise, consist or consist essentially of monomers having at least one olefinic bond, oligomers having at least one olefinic bond, polymers having at least one olefinic bond, olefins, halogenated olefins, acrylates, methacrylates, pyrroles, acrylamides, bisacrylamides, styrenes, epoxides, cyclohexeneoxide, amino acids, peptides, proteins, fatty acids, lipids, nucleotides, oligonucleotides, synthetic nucleotide analogues, nucleic acids, sugars, carbohydrates, cytokines, hormones, receptors, growth factors, drugs, and mixtures thereof.

The composition generally comprises 5-50 wt. % polymerisable precursor.

Generally, the polymer is an intrinsically conductive polymer. The polymer generally includes repeating units comprising at least one aromatic cycle, typically a 5 to 7 member aromatic cycle, optionally including one or more heteroatoms, in particular one or more N or S heteroatoms. The repeating units may include one or more N, S or O atoms outside the aromatic cycle.

The polymer generally includes repeating units comprising at least one double bond.

Typically the polymer includes repeating units comprising more than one aromatic cycle, repeating units comprising an aromatic cycle and a double bond or repeating units comprising an aromatic cycle and one or more N, S or O atoms outside the aromatic cycle.

Suitably the polymer comprises, consists or consists essentially of one or more of the group consisting of poly(fluorene)s, polyanilines, polyphenylenes, polypyrenes, polyazulenes, polynaphthalenes, Poly(acetylene)s, Poly(p-phenylene vinylene), poly(pyrrole)s, polycarbazoles, polyindoles, polyazepines, poly(thiophene)s, poly(3,4-ethylenedioxythiophene), poly(p-phenylene sulfide), polpyrenes, polyfurans.

Alternatively, the polymer may be a block copolymer with conductive blocks, hyperbranched polymers, dendrimers, supramolecular polymers, where the conductive blocks are derivatives of derivatives of pyrrole, aniline, thiophene, 3,4-ethylenedioxythiophene, fluorene, phenylene, pyrene, azulene, napthalene, indole, azepine, p-phenylene sulfide, p-phenylene vinylene, and/or furans.

According to one embodiment, the polymer may comprise, consist of or essentially consist of fullerenes, for example bucky-balls, nanotubes and nano-horns or graphenes.

The polymer is biocompatible.

Generally the polymer itself has a relatively high associated conductivity (10⁻¹⁰ S/cm to 10⁴ S/cm).

Alternatively, the composition may include a polymerisable precursor suitable to form a polymer having a relatively low electrical conductivity such as ceramics or glasses and an electrically conductive material dispersed throughout. Suitable such conductive material includes inorganic metals and alloys such as Au, Ti, Steel, CoCr, CoCrMo, Ti₆Al₄V, Ti₆Al₇Nb, iron, WE43 magnesium alloy, Mg/Zn.

The composition of the present teachings includes a polymerisable precursor and a photo-initiator.

The polymerisable precursor itself may comprise the photo-initiator, in particular, the polymeric precursor may be functionalised with photoactivatable groups.

Alternatively, or additionally, the photo-initiator may be provided separately from the polymerisable precursor.

According to one embodiment, the polymerisable precursor may be encapsulated within, or coated with the photo-initiator.

Any suitable photo-initiator may be used. Mention may be made of azo compounds, azobisisobutyronitrile, peroxides, benzoyl peroxide, aliphatic ketones and diketones, aromatic diketones, benzophenone, 9-fluorenone 2-carboxylic acid, ion pairs, the ion pair Fe3⁺OH⁻, the ion pair Pb2⁺Cl⁻), photosensitive dyes, eosin, rose Bengal, erydirosin, photosensitive transition metal derivatives, and Mn₂(CO)₁₀ in the presence of organic halides, triarylsulfonium salts with complex metal halide anions, diaryliodonium salts with complex metal halide anions, mixed arene cyclopentadienyl metal salts of complex metal halide anions, and (6-benzene)(5-cyclopentadienyl)Fe(II) hexafluorophosphate.

The composition generally comprises 1-10 wt. % photo-initiator.

The composition generally includes second photo-initiatable functional moieties which become excited at a different wavelength to the photo-initiator. Generally the difference in wavelength is at least 200 nm.

Other photo-initiation systems include, but are not limited to, redox-type photo-initiators useful in aqueous systems (e.g., ion pairs such as Fe³⁺OH⁻, and Pb²⁺Cl⁻), photosensitive dyes such as eosin, rose Bengal, and erythrosin, and transition metal derivatives such as Mn₂(CO)₁₀ in the presence of organic halides, riboflavin/triethanolamine, Irgacure 184, Irgacure 369, Irgacure 651, Irgacure 2959, azobisisobutyronitrile, peroxides such as benzoyl peroxide, aliphatic carbonyl compounds such as ketones and diketones, and aromatic diketones such as benzophenone and its derivatives, and 9-fluorenone 2-carboxylic acid.

The polymerisable precursor may comprise the second photo-initiatable functional moieties.

Generally, the composition is aqueous, although in some embodiments non-aqueous compositions may be of utility.

The composition is generally in the form of a gel, cream, liquid, suspension, solution, emulsion or foam. Typically, the composition is in the form of an injectable gel. Suitable gel based carriers include synthetic polymers (e.g. polyethylene glycol) and biopolymers (e.g. polysaccharides [hyaluronic acid, chitin, chitosan, alginate, cellulose], proteins [collagen, fibronectin, silk, etc.], lignins, polynucleotides, extracellular matrix).

The composition may suitably comprise 5-25% gel based carrier.

According to one embodiment, the composition includes particles in an injectable carrier material, said particles comprising a core having an electrical conductivity of at least 10⁻¹⁰ S/cm to 10⁴ S/cm, and a coating comprising the photo-initiator.

The composition generally comprises polymerisable precursors suitable for forming a polymer having an electrical conductivity of at least 10⁻¹⁰ S/cm, a photo-initiator and a dye.

According to a further aspect of the present invention, there is provided a buffered aqueous based composition comprising derivatives of pyrrole, aniline, thiophene, 3,4-ethylenedioxythiophene, fluorene, phenylene, pyrene, azulene, napthalene, indole, azepine, p-phenylene sulfide, p-phenylene vinylene, and/or furans, a photo-initiator, a dye.

According to a further aspect of the present invention there is provided a buffered aqueous based composition comprising pyrrole, a photo-initiator (generally irgacure) and a dye.

The present invention will now be described by way of example only, with reference to the accompanying Figures, in which:

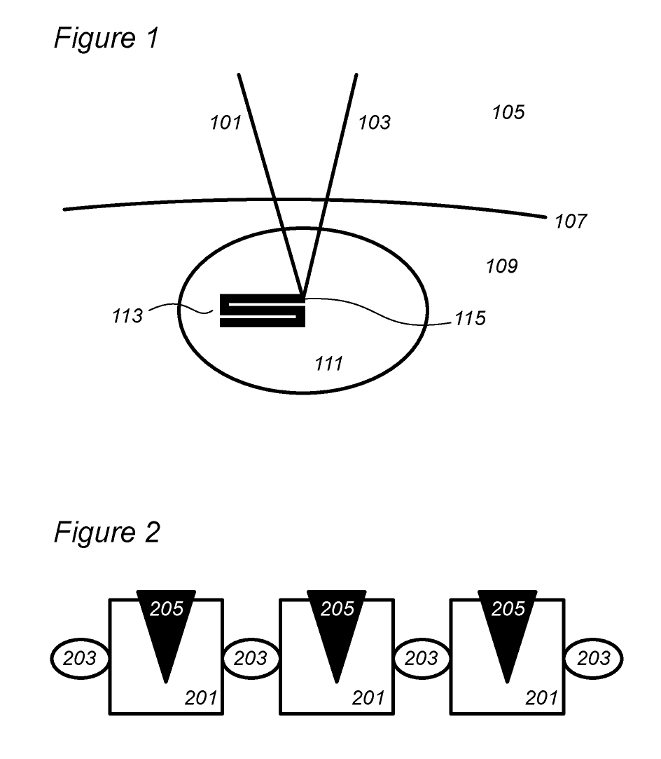

FIG. 1 provides a schematic representation of the manufacture of a subcutaneous structure according to the present invention. (not to scale);

FIG. 2 provides a schematic representation of assembly and disassembly of polymers according to the present invention;

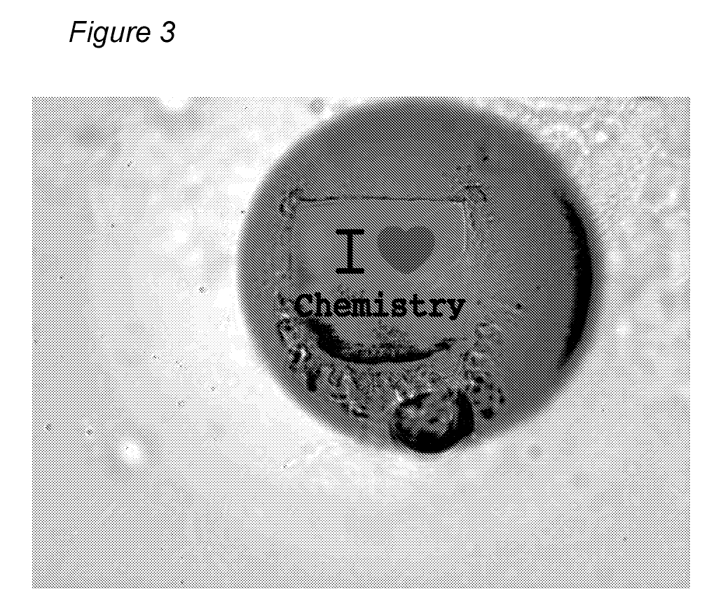

FIG. 3 is a halftone rendering of a photograph of a polymeric structure printed into vertebrate tissue.

FIG. 1 shows schematically and not to scale the manufacture of a subcutaneous device according to the present invention. A first photon beam (101) and a second photon beam (103) pass from outside (105) tissue, through the surface (107) of tissue, into the interior of tissue (109). Previously a region of precursor material (111) has been placed into the tissue (109). The photon beams cause polymerisation of precursor monomers at the point of intersection (115), and when the beams are moved, this point (115) moves and a polymerised structure (113) results.

FIG. 2 shows schematically assembly and disassembly of polymers according to the present invention. Each square block (201) represents a monomer unit that has polymerized via a multi-photon polymerisation reaction creating a bond (203) to a neighbouring monomer unit (201). In this embodiment each monomer also comprises a suitably designed moiety (205; represented by a triangle), via which a second multi-photon process may disassemble the polymer when required.

A composition of the present invention, was provided including pyrrole as the polymerisable precursor and irgacure as the photo-initiator. The composition was injected under the skin of a chicken breast. The external surface of the chicken breast was illuminated with two laser beams, and at the location of intersection, the photo-initiator was excited causing localised polymerisation of the pyrrole to form polypyrrole. The lasers were used to controllably initiate polymerisation in a targeted manner. The polypyrrole is shown in FIG. 3 as the dark lines which read “I heart Chemistry”.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A method of producing a visual marking on the exterior of a human or animal body comprising:
implanting a composition at a site in the human or animal body 1 cm or less from the exterior of the human or animal body wherein the composition comprises a polymerisable precursor and a photo-initiator wherein the photo-initiator causes the polymerisable precursor to polymerise where the photo-initiator is in its excited state,
illuminating the site with electromagnetic radiation having a wavelength of 400 to 1600 nm, wherein absorption of two or more photons of the electromagnetic radiation excites the photo-initiator causing initiation of polymerisation of the polymerisable precursor to form a polymer within the human or animal body, wherein the polymer is visible on the exterior of the human or animal body and wherein the resultant polymer has an electrical conductivity of 10⁻¹⁰ S/cm or more.

2. The method as claimed in claim 1 wherein the resultant polymer shows more than one colour on the exterior of the human or animal body.

3. The method as claimed in claim 1 wherein the visual marking is in the form of a tattoo.

4. The method as claimed in claim 1 wherein the composition is injected into the lower surfaces of the skin of the human or animal body and the visual marking may grow out as the skin is shed.

5. The method as claimed in claim 1 wherein the composition is injected under the skin of the human or animal body, to provide a permanent visual marking.

6. The method as claimed in claim 1 wherein the polymer is in the form of a solid, gel, paste or foam; generally a solid, hydrogel or plastics material.

7. The method as claimed in claim 1 wherein, the dimensions of the polymer formed (width, length, thickness) are at least 1 nm, typically at least 100 nm.

8. The method as claimed in claim 1 wherein the two or more photons are from intersecting photon beams.

9. The method as claimed claim 1 wherein the composition includes second photo-initiatable functional moieties which become excited at a second wavelength different to the first wavelength wherein the polymer may be dis-assembled through illumination of the site with electromagnetic radiation having the second wavelength, wherein absorption of two or more photons of the electromagnetic radiation causes the second photo-initiatable functional moieties on the polymer to become excited and dis-assemble the polymer.

10. The method as claimed in claim 9 wherein the second wavelength is at least 200 nm higher or lower than the first wavelength.

11. The method as claimed in claim 1 wherein the composition includes a visual indicator which alters upon polymerisation of the polymerisable precursor, wherein the visual indicator is visible in the polymerisable precursor but not in the polymer, or the visual indicator is visible in the polymer but not the polymerisable precursor.

12. The method as claimed in claim 1 wherein the composition has a viscosity of from 1 centipoise to 250,000 centipoise.

13. The method as claimed in claim 1, wherein the polymerisable precursor comprises, consists or consists essentially of monomers having at least one olefinic bond, oligomers having at least one olefinic bond, polymers having at least one olefinic bond, olefins, halogenated olefins, acrylates, methacrylates, pyrroles, acrylamides, bisacrylamides, styrenes, epoxides, cyclohexeneoxide, amino acids, peptides, proteins, fatty acids, lipids, nucleotides, oligonucleotides, synthetic nucleotide analogues, nucleic acids, sugars, carbohydrates, cytokines, hormones, receptors, growth factors, drugs, and mixtures thereof.

14. The method as claimed in claim 1 wherein the resultant polymer comprises, consists or consists essentially of one or more of the group consisting of poly(fluorene)s, polyphenylenes, polypyrenes, polyazulenes, polynaphthalenes, Poly(acetylene)s, Poly(p-phenylene vinylene), poly(pyrrole)s, polycarbazoles, polyindoles, polyazepines, poly(thiophene)s, poly(3,4-ethylenedioxythiophene), poly(p-phenylene sulfide) and polyanilines.

15. The method as claimed in claim 1 wherein the composition is in the form of a buffered aqueous based composition comprising pyrrole, a photo-initiator (generally irgacure) and a dye.
